(19) United States
(12) Patent Application Publication
(10) Pub. No.: US 2026/0337550 A1
(43) Pub. Date: Oct. 29, 2026

(54) SUCROSE NEGATIVE STREPTOCOCCUS THERMOPHILUS FOR USE IN PREPARATION OF FERMENTED PRODUCTS

(71) Applicant: Compagnie Gervais Danone, Paris (FR)

(72) Inventors: Peggy GARAULT, Saclay (FR); Jean-Michel FAURIE, Jouy-en-Josas (FR); Gaëlle QUERE, Montlhery (FR)

(21) Appl. No.: 19/445,334

(22) Filed: Jan. 9, 2026

Related U.S. Application Data
(63) Continuation of application No. 17/758,150, filed on Dec. 30, 2020, now Pat. No. 12,545,887.

(30) Foreign Application Priority Data
Dec. 30, 2019 (EP) 19306785.7

(51) Int. Cl. C12N 1/205; A23C 9/123

(52) U.S. Cl. CPC C12N 1/205; A23C 9/1238; A23C 2220/202; A23C 2260/05; A23V 2400/249

(57) ABSTRACT

The present invention relates to novel sucrose negative (Suc−) Streptococcus thermophilus strains, compositions comprising said strains, uses thereof, methods for obtaining such strains and methods for the preparation of fermented food products using such compositions.

### CROSS-REFERENCE TO RELATED APPLICATIONS

This application is a continuation of U.S. application Ser. No. 17/758,150, filed Jun. 29, 2022, which is a National Phase of PCT Application No. PCT/EP2020/088014, filed Dec. 30, 2020, which claims the benefit of EP Application No. 19306785.7, filed Dec. 30, 2019, the disclosures of each of which are hereby incorporated by reference in their entireties.

### REFERENCE TO ELECTRONIC SEQUENCE LISTING

The sequence listing of the present application is submitted electronically as an XML file named “034546.00032_ST26.xml”, created on Jan. 8, 2026 and having a size of 30,087 bytes. This sequence listing submitted electronically is an integral part of the specification and is incorporated herein by reference in its entirety.

### FIELD OF THE INVENTION

The present invention relates to novel sucrose negative (Suc−) Streptococcus thermophilus strains, compositions comprising said strains, uses thereof, methods for obtaining such strains and methods for the preparation of fermented food products.

### TECHNICAL BACKGROUND

Streptococcus thermophilus (also referred to as Streptococcus salivarius subsp. thermophilus and hereinafter also referred to as S. thermophilus) is a gram-positive lactic acid bacterium that is widely used in the preparation of fermented milk products. S. thermophilus is commonly used together with Lactobacillus delbrueckii subsp. bulgaricus (also referred to as L. bulgaricus) as a starter culture to prepare yogurt and other types of fermented milks.

Yogurts and other fermented milk products are widely consumed and contribute to a healthy diet by providing essential nutrients. However unflavoured fermented milk products have characteristic tart or acidic flavours, thus flavoured and sweetened fermented milk products are popular. Increasing consumer awareness of the benefits of a low-sugar diet together with a trend towards what the consumer perceives as “natural” products has led to a need for the development of fermented milk products with a reduced sugar content that use natural sweetening agents (e.g. stevia). The use of high sugar producing strains of S. thermophilus and L. Bulgaricus strains has also been proposed as a means of increasing the sweetness of fermented milk products.

WO 2013/160413 discloses mutant S. thermophilus strains having sweetening properties due to their secretion of glucose. These strains are characterized by a mutation in the glucokinase (glcK) gene and are described as being suitable for use in the preparation of fermented milk products. However, it is also disclosed that for such strains the fermentation process is delayed by 2-5 hours requiring at least 20 hours of fermentation. Sorensen et al. (Appl Environ Microbiol. 2016 Apr. 22) also disclose glucose secreting S. thermophilus strains St1-GS-1; St1-GS-2; St2-GS-1; St2-GS-2. Strains St1-GS-2 and St2-GS-2 could not acidify milk and glucokinase mutant strains (i.e. St1-GS-1 and St2-GS-1) displayed a lag of 2-3 hours over the parent strains in onset of acidification.

WO 2011/026863 discloses highly texturizing S. thermophilus strains with mutations in the galK gene that produce a high viscosity fermented milk. WO 2017/103051 discloses S. thermophilus strains with enhanced properties for sweetening of food products in combination with improved texturizing properties.

Nevertheless, it remains challenging to reduce the amount of sugar required for the preparation of fermented products while ensuring a product having good organoleptic properties, low amounts of added sugar and having a so-called “clean label” (a short ingredient list, using as many consumer recognizable ingredients as possible). Moreover, the acidity of fermented products can also be increased after the lactic fermentation by post acidification due to the consumption of other sugars by the bacteria. Consequently, it is sometimes necessary to use an increased quantity of sugar in the original recipe or to add sugar after the fermentation.

There thus remains a need for means for the preparation of fermented products using a reduced amount of sucrose ingredients while having good organoleptic qualities.

### SUMMARY OF THE INVENTION

The present invention follows from the hypothesis formulated by the Inventors that S. thermophilus strains rendered deficient in sucrose metabolism could reduce the amount of sugar required in the preparation of fermented food products such as fermented milk products and limit undesired post-acidification, while maintaining the properties of said strains necessary for the preparation of fermented food products, e.g. the capacity to metabolize lactose in order to effectively ferment dairy milk and the capacity to produce exopolysaccharides (EPS) in order to texturize the fermented product. However, the selection of such strains has been proved difficult and significant attempts made by the Inventors using a screening strategy failed to isolate sucrose negative (Suc−) variants of high-performance S. thermophilus strains.

Unexpectedly, the Inventors discovered that such variants can be obtained by selecting galactose positive (Gal+) variants prior to the selection of Suc− variants. Accordingly, the present invention provides Suc− Gal+ Streptococcus thermophilus strains. The present invention also provides compositions comprising said strains, uses thereof, methods for obtaining such strains and methods for the preparation of fermented food products using such compositions.

### DETAILED DESCRIPTION OF THE INVENTION

As used herein the term “sucrose negative” (or Suc−) means the inability of a bacterium to metabolize sucrose. According to the present invention, a Suc− S. thermophilus strain is not able to grow in a medium containing sucrose as the sole source of carbon. Conversely, the term “sucrose positive” (or Suc+) means the ability of a bacterium to metabolize sucrose or to grow in a medium containing sucrose as a sole source of carbon. These phenotypes can be determined using techniques well known by a skilled person in the art. For example, these phenotypes can be assessed by inoculating—into a M17 broth containing 2% sucrose—an overnight culture of the S. thermophilus strain to be tested at a rate of 1%, and incubating for hours at 37 °C, wherein a pH of 6 or above at the end of incubation is indicative of a Suc− phenotype and a pH of 5.5 or lower at the end of incubation is indicative of a Suc+ phenotype. Alternatively the metabolic profile of a bacterial strain can be determined using commercially available kits such as the API gallery (bioMerieux).

The above definition also applies to the phenotypes related to other carbohydrates fermentable by lactic acid bacteria, in particular lactose, galactose, glucose or fructose.

Accordingly, the terms “Lactose negative” (or Lac−), “galactose negative” (or Gal−), “glucose negative” (or Glu−) or “fructose negative” (or Fru−) refer to the inability of a bacterium to metabolize lactose, galactose, glucose or fructose respectively. Conversely, the terms “Lactose positive” (or Lac+), “galactose positive” (or Gal+), “glucose positive” (or Glu+) or “fructose positive” (or Fru+) refer to the ability of a bacterium to metabolize lactose, galactose, glucose or fructose respectively. As detailed above, these phenotypes can be easily assessed by testing growth of S. thermophilus in a medium containing the sugar of interest as a sole source of carbon.

As used herein the term “fermentation” shall be taken to mean the metabolism of a substance by microorganisms, e.g. bacteria, yeasts, or other microorganisms. As used herein the term “highly fermentative” as used in reference to lactic acid bacteria shall be taken to mean a strain thereof that is capable of significantly reducing the pH of a suitable medium at a suitable culture temperature. In regards to a S. thermophilus strain, said strain shall be capable of reducing the pH of a media comprising semi-skimmed cow milk supplemented with yeast extract (2 g/l) when inoculated with a 1% volume inoculum by at least 1.8 pH points (e.g. from 6.5 to 4.7) in less than 10 hours, more preferably less than 9, 8, 7, 6, 5 or 4 hours when cultured at a temperature of about 37 °C to about 43 °C, more preferably 37 °C to 43 °C. Said inoculum comprises about 10⁵ cfu to about 10⁷ cfu of said S. thermophilus per gram of inoculum composition, more preferably about 10⁶ cfu to about 10⁷ cfu.

As used herein, the term “texturizing” means that a S. thermophilus strain is able to significantly increase the viscosity of a fermented product by the production of exopolysaccharides (EPS). Methods for determining the texture of milk include measuring the shear stress (viscosity) of the fermented milk and are readily known in the art and also described in the examples below.

As used herein the term “stable composition” shall be taken to mean a composition that does not present sedimentation and/or serum separation.

As used herein the term “x % (w/w)” is equivalent to “x g per 100 g”.

As used herein the terms “dairy composition”, “milk-based composition” or “dairy product” shall be taken to mean a product or composition comprising essentially of or consisting of milk or milk components and optionally further ingredients.

As used herein the term “fermented dairy” shall be taken to mean a product or composition that is the product of the acidifying fermentation of a milk-based composition by a starter culture of fermenting microorganisms, in particular bacteria, preferably lactic acid bacteria. As used herein the term “fermented milk” shall be taken to mean a product or composition derived from milk by the acidifying action of at least one lactic acid bacterium.

Accordingly, as used herein a fermented dairy product can thus be a fermented milk, such as a yoghurt (e.g. a set, stirred or drink yogurt), or a fresh cheese such as a white cheese or a “petit-Suisse”. It can be also be a strained fermented milk such as a strained yoghurt (e.g. a concentrated or Greek-style yoghurt).

The terms “fermented milk” and “yogurt” or “yoghurt” are given their usual meanings in the field of the dairy industry, that is, products suitable for human consumption and originating from acidifying lactic fermentation of a milk substrate. These products can contain secondary ingredients such as fruits, vegetables, sugar, etc. The expression “fermented milk” may be used to refer to fermented milks other than yogurts e.g. “Kefir”, “Kumtss”, “Lassi”, “Dahi”, “Leben”, “Filmjolk”, “Villi”, “Acidophilus milk”.

The term “yogurt” or “yoghurt” as used herein shall be taken to mean fermented milk obtained by the acidifying lactic fermentation of specific thermophilic lactic acid bacteria such as Lactobacillus delbrueckii subsp. bulgaricus and Streptococcus thermophilus (also referred to as Streptococcus salivarius subsp. thermophilus), which must be in the living state in the finished product at a minimum CFU. In certain countries, regulations allow the addition of further lactic acid bacteria to yoghurt such as but not limited to strains of Bifidobacterium and/or Lactobacillus acidophilus and/or Lactobacillus casei. These additional lactic acid bacteria strains are intended to impart various properties to the finished product, such as that of providing organoleptic qualities, favoring equilibrium of intestinal flora or modulating the immune system.

As used herein the term “strained fermented dairy composition” shall be taken to mean a fermented dairy composition which has been subjected to a post-fermentation acid whey separation process.

As used herein the term “spoonable” shall be taken to mean a solid or semi-solid that may be consumed by means of a spoon or other utensil.

As used herein the term “cfu” or “CFU” shall be taken to be an abbreviation of the term “colony forming unit”.

As used herein the term “CNCM I−” followed by a 4 digit number shall be taken to refer to a strain deposited at the Collection Nationale de Cultures de Microorganismes (CNCM) 25 rue du Docteur Roux, Paris, France under the Budapest Treaty with an accession number corresponding to said 4 digit number, e.g. CNCM I-5448.

As used herein reference to a bacterial strain or species shall be taken to include functionally equivalent bacteria derived therefrom such as but not limited to mutants or variants. These mutants or variants can be strains wherein one or more endogenous gene(s) of the parent strain has (have) been mutated by non-targeted means (random mutagenesis induced e.g. by UV radiation or alkylating agents), for instance to modify some of their metabolic properties (e.g., their ability to ferment sugars, their resistance to acidity, their survival to transport in the gastrointestinal tract, their post-acidification properties or their metabolite production).

As used herein the term “GMO” shall be taken to mean an organism, with the exception of human beings, in which the genetic material has been altered in a way that does not occur naturally by mating and/or natural recombination, said term shall be taken to encompass for example organisms that are the subject of genetic transformation to include exogenous DNA, or site targeted gene editing such as CRISPR/Cas9 methodologies. The term “GMO” shall be taken to exclude and not encompass an organism in which the genetic material has been altered by random mutagenesis such as by chemical or radiation means followed by a phenotypic selection.

In the present text, strains, mutants and variants derived from a parent species or strain will be considered as being encompassed by reference to said parent species or strain, e.g. the phrases “Streptococcus thermophilus” and “CNCM I-5448” shall be taken to include strains, mutants and variants derived therefrom. Accordingly, as used herein reference to a bacterial strain specified by an accession or deposit number shall be taken to encompass variants thereof having at least 95% identity (see: Stackebrandt & Goebel, 1994, Int. J. Syst. Bacteriol. 44:846-849). In a particularly preferred embodiment, said variant has at least 97% identity with the 16S rRNA sequence of said specified strain, more preferably at least 98% identity, more preferably at least 99% or more identity.

As used herein the term “substantially pure” when used in reference to a bacterial strain refers to the percent of said bacterial strain relative to the total micro-organism content. Substantially pure can be at least about 99.99%, at least about 99.90%, at least about 99.50%, at least about 99.00%, at least about 95.00%, at least about 90.00%, at least about 85.00%, or at least about 75.00%.

As used herein, a “lactic acid bacterium” is a Gram-positive, acid-tolerant, generally non-sporulating and non-respiring, either rod- or cocci-shaped bacterium that is able to ferment sugars into lactic acid.

Streptococcus thermophilus

In an aspect, the present invention provides a strain of Streptococcus thermophilus characterized in that it is galactose positive (Gal+) and sucrose negative (Suc−).

In an embodiment, the present invention provides a strain as defined above further characterized in that it is fructose negative (Fru−).

In an embodiment, the present invention provides a strain as defined above further characterized in that it is glucose positive (Glu+).

In an embodiment, the present invention provides a strain as defined above further characterized in that it is lactose positive (Lac+).

In a preferred embodiment, the present invention provides a strain of Streptococcus thermophilus characterized in that it is Gal+ Suc− Fru−, preferably Gal+ Suc− Fru− Glu+, most preferably Gal+ Suc− Fru− Glu+Lac+.

Such strains are particularly appropriate for the preparation of flavored fermented products since they limit or avoid the decrease of sucrose and/or fructose present in the original mixture.

In a most preferred embodiment, the present invention provides a strain of Streptococcus thermophilus as defined above characterized in that it is highly fermentative.

In a preferred embodiment, the present invention provides a strain as defined above further characterized in that it is texturizing.

In a preferred embodiment, the present invention provides a strain as defined above further characterized in that it is non-GMO organism with the exception of a variant of a naturally occurring strain that has been derived by a process of random mutagenesis by chemical or radiation means and phenotypic selection. Thus for example a strain of the invention may be a galactose positive (Gal+) and sucrose negative (Suc−) derived from a wild type S. thermophilus strain by means of UV radiation or alkylating agent random mutagenesis followed by a selection based on metabolic profile.

In an embodiment, the strain of the invention produces exopolysaccharide when cultured in a milk based medium, and in preferred embodiments, may be used to prepare a fermented milk product having a viscosity greater than 800 mPa·s, more preferably greater than 900 mPa·s and most preferably greater than 1000 mPa·s, at 4 °C. In embodiments, the composition has a viscosity range of 800 to 2000 mPa·s, or 800 to 1500 mPa·s, at 4 °C. Said viscosity being measured using a refrigerated MCR301 rheometer equipped with a 27 mm concentric cylinder system according to equation I after 7 days of storage at 4 °C.

In an embodiment, the present invention provides a strain of Streptococcus thermophilus characterized in that its genome comprises at least one mutation selected from the group consisting of a substitution in the lacS gene corresponding to SEQ ID NO: 1, a substitution in the galK promoter, preferably in the Cre site corresponding to TGAAAGGGGTTACG (SEQ ID NO: 15), a non-sense mutation in the scrA gene corresponding to SEQ ID NO: 7, and a substitution in the galR gene corresponding to SEQ ID NO: 11.

In an embodiment, the genome of a strain according to the invention comprises two, three or all mutations from the group consisting of a substitution in the lacS gene corresponding to SEQ ID NO: 1, a substitution in the galK promoter, preferably in the Cre site corresponding to SEQ ID NO: 15, a non-sense mutation in the scrA gene corresponding to SEQ ID NO: 7, and a substitution in the galR gene corresponding to SEQ ID NO: 11.

In an embodiment, the substitution in the lacS gene is a substitution, in particular C/A, in position 1159 of SEQ ID NO: 1. In an embodiment, the substitution in the galK promoter is a substitution, in particular G/T, in position 228 of SEQ ID NO: 5. In an embodiment, the non-sense mutation in the scrA is a substitution, in particular C/T, in position 1393 of SEQ ID NO: 7. In an embodiment, the substitution in the ga/R gene is a substitution, in particular T/C, in position 281 of SEQ ID NO: 11.

In an embodiment, the genome of a strain according to the invention comprises a substitution, in particular C/A, in position 1159 of SEQ ID NO: 1, a substitution, in particular G/T, in position 228 of SEQ ID NO: 5 and a substitution, in particular C/T, in position 1393 of SEQ ID NO: 7.

In an embodiment, the present invention provides a strain of Streptococcus thermophilus characterized in that its genome comprises at least one sequence selected from the group consisting of: SEQ ID NO: 2, SEQ ID NO: 6, SEQ ID NO: 8 and SEQ ID NO: 12.

In an embodiment, the present invention provides a strain of Streptococcus thermophilus characterized in that its genome comprises two, three or all sequences selected from the group consisting of: SEQ ID NO: 2, SEQ ID NO: 6, SEQ ID NO: 8 and SEQ ID NO: 12.

In an embodiment, the present invention provides a strain of Streptococcus thermophilus characterized in that its genome comprises the sequences SEQ ID NO: 2, SEQ ID NO: 6 and SEQ ID NO: 8.

In an embodiment, the present invention provides a strain of Streptococcus thermophilus characterized in that it expresses at least one protein selected from the group consisting of a LacS protein of SEQ ID NO: 4, a ScrA protein of SEQ ID NO: 10 and a GalR protein of SEQ ID NO: 14.

In an embodiment, the present invention provides a strain of Streptococcus thermophilus characterized in that it expresses two, three or all proteins selected from the group consisting of a LacS protein of SEQ ID NO: 4, a ScrA protein of SEQ ID NO: 10 and a GalR protein of SEQ ID NO: 14.

In an embodiment, the present invention provides a strain of Streptococcus thermophilus characterized in that it expresses a LacS protein of SEQ ID NO: 4, a ScrA protein of SEQ ID NO: 10.

In an embodiment, the present invention provides the strain Streptococcus thermophilus CNCM I-5448.This strain has been deposited at the Collection Nationale de Cultures de Microorganismes (CNCM) (Institut Pasteur, 25 Rue du Docteur Roux, Paris, France) under the Budapest Treaty on 20^{th} November 2019 under reference number CNCM I-5448. The deposit was made in accordance with the Budapest Treaty on the International Recognition of the Deposit of Microorganisms for the Purposes of Patent Procedure, as provided therein the applicant requests that a sample of the deposited micro-organisms only be made available to an independent expert, until the date on which the patent may be granted. The strain S. thermophilus CNCM I-5448 corresponds to clone 11R15 described in the examples below.

In an embodiment, the present invention provides the strain S. thermophilus CNCM I-5448, that is substantially pure.

### Methods for Obtaining Sucrose Negative Strains

Most wild type S. thermophilus strains are sucrose positive and to the development of sucrose negative variants by random mutagenesis has proven challenging. Further, the metabolic pathway of carbohydrates and the biosynthesis pathway of the exopolysaccharides are closely linked in S. thermophilus and a deleterious mutation in the sucrose metabolism drastically reduces the growth of S. thermophilus. However, as shown in the examples below, this difficulty of directly isolating Suc− variants is overcome by isolating Gal+variants prior to the selection of Suc− variants.

Most wild type S. thermophilus strains are Gal- and almost all texturizing strains contain galactose in the EPS. Without wishing to be bound by theory, it was thus hypothesized that the galactose pathway could compensate for a deficiency in the sucrose pathway and allow the isolation of Suc− variants that also retain the texturizing capacity of the wild-type strain.

Accordingly, an aspect of the present invention relates to a method for obtaining a sucrose negative S. thermophilus strain comprising:
i) providing a first S. thermophilus strain,
ii) selecting at least one galactose positive clone thereof,
iii) mutating said selected clone(s) selected in ii), preferably by means of chemical or radiation and selecting at least one sucrose negative variant thereof.

In an embodiment, said first S. thermophilus strain is Gal− Suc+.

In a preferred embodiment, said first S. thermophilus strain is Gal− Suc+Fru−.

In a most preferred embodiment, said first S. thermophilus strain is Gal− Suc+Fru− Glu+Lac+.

In an embodiment, said first S. thermophilus strain is an exopolysacharride producing texturizing strain.

Step ii) can be carried out by, but is not limited to, subculturing the first S. thermophilus strain in a medium with galactose as the sole carbon source, preferably in M17 medium, most preferably in a modified M17 medium (glycerophosphate 9 g/l).

Step iii) is preferably carried out by random mutagenesis, preferably by exposure to alkylating agents or UV exposure, more preferably by exposure to a dose of UV giving approximately 2% survival.

In an embodiment, said method further comprises:
iv) selecting a sucrose negative variant that is highly fermentative in a milk based medium and/or subculturing a sucrose negative variant in a milk based medium. This additional step allows the isolation at least one strain having faster milk fermentation capabilities.

Subculturing of a sucrose negative variant in a milk based medium is preferably carried out more than one time, for example 2, 3, 4, 5, 6, 7, 8, 9, 10, 11, 12, 13, 14 or 15 times. Preferably, subculturing is carried out at least 10 times, more preferably at least 15 times.

In an embodiment, step iii) is carried out by mutating the lacS gene corresponding to SEQ ID NO: 1, and/or the galK promoter, preferably in the Cre site corresponding to TGAAAGGGGTTACG (SEQ ID NO: 15), and/or the scrA gene corresponding to SEQ ID NO: 7, and/or the galR gene corresponding to SEQ ID NO: 11.

In an embodiment, step iii) is carried out by introducing a substitution in the lacS gene corresponding to SEQ ID NO: 1, and/or a substitution in the galK promoter, preferably in the Cre site corresponding to TGAAAGGGGTTACG (SEQ ID NO: 15), and/or a non-sense mutation in the ScrA gene corresponding to SEQ ID NO: 7, and/or a substitution in the galR gene corresponding to SEQ ID NO: 11.

In an embodiment, the substitution in the lacS gene is a substitution, in particular C/A, in position 1159 of SEQ ID NO: 1. In an embodiment, the substitution in the galK promoter is a substitution, in particular G/T, in position 228 of SEQ ID NO: 5. In an embodiment, the non-sense mutation in the scrA is a substitution, in particular C/T, in position 1393 of SEQ ID NO: 7. In an embodiment, the substitution in the ga/R gene is a substitution, in particular T/C, in position 281 of SEQ ID NO: 11.

### Compositions of the Invention

In an aspect, the present invention provides compositions comprising a Streptococcus thermophilus strain as defined above.

Preferably, the composition comprises at least 10⁵, more preferably at least 10⁶ and most preferably at least 10⁷ colony forming unit (CFU) of a S. thermophilus strain of the invention per gram (g) of composition.

In embodiments, the composition comprises from 105 to 10¹² CFU of a S. thermophilus strain of the invention per gram (g) of composition. In embodiments, the composition comprises 10⁶ to 10¹¹ CFU of a S. thermophilus strain of the invention per gram (g) of composition. In embodiments, the composition comprises at least 10⁵, 10⁶, 10⁷, 10⁸, 10⁹, 10¹⁰, 10¹¹ or 10¹² CFU of a S. thermophilus strain of the invention per gram (g) of composition.

In an embodiment, the composition comprises at least 10⁵, 10⁶, 10⁷, 10⁸, 10⁹, 10¹⁰, 10¹¹ or 10¹² CFU of the strain Streptococcus thermophilus CNCM I-5448 per gram (g) of composition.

The bacterium as provided herein is suitable for use in edible compositions, accordingly in one embodiment the present invention provides a composition suitable for human consumption or ingestion, preferably by oral means. Accordingly, the composition comprises or consists of comestible matter. It is particularly preferred that the compositions of embodiments of the invention are substantially free of pathogenic or toxicogenic matter. The composition according to embodiments of the invention may be a medicament or pharmaceutical composition. In a particularly preferred embodiment, the composition according to the invention may be a non-therapeutic composition, preferably a nutraceutical composition, a nutritional composition and/or a food composition. It is particularly preferred that the food composition is a fermented food composition, preferably a fermented dairy composition. Further compositions according to embodiments of the invention also include food additives, food ingredients, nutritional supplements, nutritional formulas, baby foods, infant milk formulas and infant follow-on formulas.

The composition may comprise further additional strains of Bifidobacterium and/or lactic acid bacteria; typically 1, 2, 3, 4 or more additional strains. Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum. Examples of lactic acid bacteria that can be used include but are not limited to Lactobacilli (for example Lactobacillus acidophilus, Lactobacillus buchneri, Lactobacillus delbrueckii, in particular L. delbrueckii subsp. bulgaricus or lactis or delbrueckii, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Preferably the composition further comprises Lactobacillus and/or Streptococcus. For the preparation of yogurt, the composition typically comprises Lactobacillus bulgaricus (also referred to as Lactobacillus delbrueckii subsp. bulgaricus) and Streptococcus thermophilus, optionally with additional microorganisms such as but not limited to probiotic species or other species that may provide desirable organoleptic or other qualities to the composition, e.g. further strains of Lactococcus lactis.

Accordingly, in one embodiment the present invention provides a composition comprising the strain Streptococcus thermophilus CNCM I-5448 and further comprising at least one strain of Lactobacillus bulgaricus and optionally one or more strains of Lactococcus lactis and/or Bifidobacterium.

### Dairy Compositions

The strains of the present invention are particularly suited to the preparation of fermented compositions. Accordingly, in an embodiment the present invention provides a dairy composition, preferably a fermented dairy composition. The dairy composition of the invention comprises milk, preferably fermented milk. Preferably the composition comprises at least about 30% (w/w) milk, more preferably at least about 50% (w/w) milk and even more preferably at least about 70% (w/w) milk. In embodiments, the composition comprises at 30% to 100% (w/w) milk. In embodiments, the composition comprises 50% to 100% (w/w) milk. In embodiments, the composition comprises 70% to 100% (w/w) milk. Preferably said milk is vegetal and/or animal milk, more preferably soya, almond, oat, hemp, spelt, coconut, rice, goat, ewe, camel, mare or cow milk, and most preferably to cow milk. Preferably said milk(s) are heat-treated, typically pasteurized, to ensure sterility. Preferably said heat treatment is carried out prior to the preparation of the fermented dairy composition.

Preferably, said milk comprises one or more of skimmed, partially-skimmed or non-skimmed milk. Preferably said milk or milks may be in liquid, powdered and/or concentrated form. In one embodiment said milk further comprises milk components preferably selected from the group consisting of cream, casein, caseinate (for example calcium or sodium caseinate), whey proteins notably in the form of a concentrate (WPC), milk proteins notably in the form of a concentrate (MPC), milk protein hydrolysates, and mixtures thereof. In one embodiment said mixture further comprises plant and/or fruit juices. In one embodiment said milk or milks may be enriched or fortified with further milk components or other nutrients such as but not limited to vitamins, minerals, trace elements or other micronutrients.

Preferably, the dairy composition comprises above about 0.3 g per 100 g by weight free lactic acid, more preferably above about 0.7 g or 0.6 g per 100 g by weight free lactic acid. In embodiments, the composition comprises 0.3 g to 0.7 grams per 100 g by weight free lactic acid.

Preferably, the dairy composition comprises a protein content at least equivalent to that of the milk or milks from which it is derived, preferably at least about 2.5%, more preferably at least about 3% or 3.5% (w/w). Preferably the composition has a pH equal to or lower than 5, preferably between about 3 and about 4.5 and more preferably between about 3.5 and about 4.5.

Preferably, the fermented dairy composition after 7 days of storage at 4 °C has a viscosity greater than 800 mPa·s, more preferably greater than 900 mPa·s and most preferably greater than 1000 mPa·s, at 4 °C, according to equation I. In embodiments, the composition has a viscosity range of 800 to 2000 mPa·s, or 800 to 1500 mPa·s, at 4 °C, as measured using a refrigerated MCR301 rheometer equipped with a 27 mm concentric cylinder system according to equation I.

The fermented dairy composition according to embodiments of the invention is preferably a product selected from the group comprising yogurt, set yogurt, stirred yogurt, pourable yogurt, yogurt drink, frozen yogurt, kefir, buttermilk, quark, sour cream, fresh cheese and cheese. In one embodiment the composition according to embodiments of the invention is a drinkable composition, more preferably a fermented milk drink such as but not limited to a yogurt drink, kefir etc. In an alternative embodiment the composition according to embodiments of the invention is a composition that is spoonable, such as a set or stirred yogurt or equivalent thereof.

In an embodiment the fermented dairy composition is a strained fermented dairy composition. The strained fermented dairy composition preferably has the following contents (% by weight):
from 8.5% to 11.0% of milk protein
from 0.0% to 8.0% of fat, for example from 0.0% to 3.5% or from 3.5% to 8.0%
from 0.00% to 7.00% of lactose, for example from 2.80% to 6.00% The pH of the strained fermented dairy composition can for example be of from 3.80 to 4.65.

Preferably the composition, according to embodiments of the invention, may be stored, transported and/or distributed at a temperature of from 1 °C to 10 °C for at least about 30 days, at least about 60 days or at least about 90 days from packaging and remain suitable for consumption.

In embodiments, the dairy compositions of the invention comprise at least 10⁵ cfu/g, more preferably at least 10⁶ cfu/g, such as at least 10⁷ cfu/g, e.g. at least 10⁸ cfu/g, such as at least 10⁹ cfu/g, e.g. at least 10¹⁰ cfu/g, such as at least 10¹¹ cfu/g of a S. thermophilus strain of the invention per gram of dairy composition. In embodiments, the compositions of the invention comprise 10⁵ to 10¹² or 10⁶ to 10¹⁰ colony forming unit (CFU) of a S. thermophilus strain of the invention per gram of composition, preferably S. thermophilus CNCM I-5448.

Preferably, the composition is a packaged product that comprises at least 10⁶, more preferably at least 10⁷ and most preferably at least 10⁸ colony forming unit (CFU) of Streptococcus thermophilus CNCM I-5448 per gram (g) of composition according to embodiments of the invention subsequent to storage, transport and/or distribution at a temperature of from 1 °C to 10 °C for at least about 30 days, at least about 60 days or at least about 90 days from packaging.

In embodiments, the composition is a packaged product that comprises 10⁵ to 10¹² or 10⁶ to 10¹⁰ colony forming unit (CFU) of Streptococcus thermophilus CNCM I-5448 per gram (g) of composition according to embodiments of the invention subsequent to storage, transport and/or distribution at a temperature of from 1 °C to 10 °C for at least about 30 days, at least about 60 days or at least about 90 days from packaging.

In embodiments, the dairy composition further comprises an intermediate preparation. Intermediate preparations are known to the one skilled in the art. They are typically used to modify the taste, mouthfeel and/or texture of a dairy composition, for example of a fermented dairy composition. They can used also to introduce some additives such as nutrients. They typically comprise sweetening agents, flavors, color modifiers, cereals and/or fruit.

Intermediate fruit preparations are for example slurries or fruit preparations. Flavors include for example fruit flavors, vanilla flavors, caramel flavors, coffee flavors, chocolate flavors.

Fruit preparations typically comprise fruits, as used herein the term “fruit” refers to any fruit form, including for example full fruits, pieces, purees, concentrates, juices etc.

The intermediate preparation or slurry typically comprises a stabilizing agent, having at least one stabilizer. The stabilizing agent can comprise at least two stabilizers. Such stabilizers are known to the one skilled in the art. They typically help in avoiding phase separation of solids, for examples of fruits or fruits extracts and/or in avoiding syneresis. They typically provide some viscosity to the composition, for example a viscosity (Bostwick viscosity at 20 °C) of from 1 to 20 cm/min, preferably of from 4 to 12 cm/min.

The stabilizing system or the stabilizer can for example be a starch, a pectin, agar, a xanthan, a carrageenan, a locust bean gum, or a mixture thereof. The amount of stabilizing system is typically of from 0.5 to 5% by weight.

The intermediate preparation can typically comprise organoleptic modifiers. Such ingredients are known by the one skilled in the art.

The organoleptic modifiers can be for example sweetening agents different from sugar, coloring agents, cereals and/or cereal extracts.

Examples of sweetening agents are ingredients referred to as High Intensity Sweeteners, such as sucralose, acesulfam K, aspartam, saccharine.

Examples of fruits include for example strawberry, peach, apricot, mango, apple, pear, raspberry, blueberry, blackberry, passion, cherry, and mixtures or associations thereof, such as peach-passion.

The fruits can be for example provided as:
frozen fruit cubes, for example 10 mm fruit cubes, for example Individual Quick Frozen fruit cubes, for example strawberry, peach, apricot, mango, apple, pear fruit cubes or mixtures thereof,
Aseptic fruit cubes, for example 10 mm fruit cubes, for example strawberry, peach, apricot, mango, apple or pear fruit cubes or mixtures thereof,
fruit purees, for example fruit purees concentrated from 2 to 5 times, preferably 3 times, for example aseptic fruit purees, for example strawberry, peach, apricot, mango, raspberry, blueberry or apple fruit purees or mixtures thereof,
single aseptic fruit purees, for example strawberry, raspberry, peach, apricot, blueberry or apple single aseptic fruit purees or mixture thereof,
frozen whole fruits, for example Individual Quick Frozen whole fruits, for example blueberry, raspberry or blackberry frozen whole fruits, or mixtures thereof,
mixtures thereof.

The ingredients and/or components of the intermediate preparation and the amounts thereof can be typically such that the composition has a brix degree of from 1 to 65 brix, for example from 1 to 10 brix, or from 10 to 15 brix, or from 15 to 20 brix, or from 20 to 25 brix, or from 25 to 30 brix, or from 30 to 35 brix, or from 35 to 40 brix, or from 40 to 45 brix, or from to 50 brix, or from 50 to 55 brix, or from 55 to 60 brix, or from 55 to 60 brix, or from 60 to 65 brix.

A fruit preparation can for example comprise fruit in an amount of from 30% to 80% by weight, for example from 50 to 70% by weight.

The intermediate preparation can comprise water. It is mentioned that a part of the water can come from ingredients used to prepare the fruit preparation, for example from fruits or fruit extracts or from a phosphoric acid solution.

The fruit preparation can comprise pH modification agents such as citric acid. The fruit preparation can have a pH of from 2.5 to 5, preferably of from 2.8 to 4.2.

Typically a fruit preparation can be added in an amount of 5-35% by weight with reference to the total amount of composition. In embodiments the composition of the invention comprises up to about 30% (w/w) of said intermediate preparation, e.g. up to about 10%, 15%, 20%, 25% (w/w). In one embodiment, the composition according to embodiments of the invention comprise 1% to 30% (w/w) of said intermediate preparation. In alternative embodiments, the composition according to embodiments of the invention comprise 1% to 25% (w/w) of said intermediate preparation. In further alternative embodiments, the composition according to embodiments of the invention comprise 1% to 20% (w/w) of said intermediate preparation. In additional embodiments, the composition according to embodiments of the invention comprise 1% to 15% (w/w) of said intermediate preparation. In further additional embodiments, the composition according to embodiments of the invention comprise 1% to 10% (w/w) of said intermediate preparation.

Preferably the composition, according to embodiments of the invention is provided in a sealed or sealable container containing about 50 g, 60 g, 70 g, 75 g, 80 g, 85 g, 90 g, 95 g, 100 g, 105 g, 110 g, 115 g, 120 g, 125 g, 130 g, 135 g, 140 g, 145 g, 150 g, 200 g, 300 g, 320 g or 500 g or about 1 oz, 2 oz, 3 oz, 4 oz, 5 oz, 6 oz or 12 oz product by weight.

In embodiments, the composition, according to embodiments of the invention is provided in a sealed or sealable container containing about 50 g to 500 g, 60 g to 500 g, 70 g to 500 g, 75 g to 500 g, 80 g to 500 g, 85 g to 500 g, 90 g to 500 g, 95 g to 500 g, 100 g to 500 g, 105 g to 500 g, 110 g to 500 g, 115 g to 500 g, 120 g to 500 g, 125 g to 500 g, 130 g to 500 g, 135 g to 500 g, 140 g to 500 g, 145 g to 500 g, 150 g to 500 g, 200 g to 500 g, 300 g to 500 g, 320 g to 500 g or 500 g product by weight. In embodiments, the composition, according to embodiments of the invention is provided in a sealed or sealable container containing about 1 oz to 12 oz, 2 oz to 12 oz, 3 oz to 12 oz, 4 oz to 12 oz, 5 oz to 12 oz, 6 oz to 12 oz or 12 oz product by weight.

### Inoculum Compositions

The Streptococcus thermophilus strain of the invention is useful as starter culture in the preparation of food compositions, such as fermented dairy products. Accordingly, in an embodiment, the present invention provides an inoculum comprising a strain of S. thermophilus as defined above that is suitable for the preparation of fermented dairy products. The inoculum of the invention is suitable for the direct inoculation of S. thermophilus strains into a composition comprising milk to provide fermented dairy products of the invention, typically without the need for a culture step prior to the said direct inoculation. In a preferred embodiment, the present invention provides an inoculum comprising the strain S. thermophilus CNCM I-5448.

Typically the inoculum further comprises excipient or carriers, the selection of which is within the scope of the skilled person but may include buffers or culture media. The inoculum may optionally comprise further components such as cryoprotectants, preservatives and/or additives including nutrients such as yeast extracts, cysteine, sugars and vitamins.

Typically the inoculum is for use in the preparation of fermented dairy products, according in one embodiment the inoculum of the invention may be provided to the dairy composition in quantities of up to about 500 mg/l.

Typically the inoculum is fresh, frozen, dried or lyophilized. The inoculum may be in liquid, dry, spray-dried or solid form. It is particularly preferred that the inoculum is in liquid form. The inoculum may be defrosted and/or dispersed in liquid (e.g. water) prior to inoculation into a composition comprising milk.

In embodiments, the inoculum comprises at least 10⁶ cfu, at least 10⁷ cfu, at least 10⁸ cfu, 10⁹ cfu, e.g. at least 10¹⁰ cfu, such as at least 10¹¹ cfu of a S. thermophilus of the invention per gram of inoculum composition. In embodiments, the inoculum comprises 10⁷ to 10¹² colony forming unit (CFU), or more preferably 10¹⁰ to 10¹² colony forming unit (CFU) of a S. thermophilus strain of the invention per gram of inoculum.

Preferably, the inoculum comprising a S. thermophilus strain of the invention is substantially pure.

In a further embodiment, the present invention provides a mixture or kit of parts of the inoculum of the invention together with inoculum of Bifidobacterium and/or lactic acid bacteria.

Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum. Examples of lactic acid bacteria that can be used include but are not limited to Lactobacilli (for example Lactobacillus acidophilus, Lactobacillus buchneri, Lactobacillus delbrueckii, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Preferably the inoculum mixture further comprises Lactobacillus and/or Streptococcus. For the preparation of yogurt, the inoculum mixture typically comprises Lactobacillus bulgaricus (also referred to as Lactobacillus delbrueckii subsp. bulgaricus) and Streptococcus thermophilus, optionally with additional microorganisms such as but not limited to probiotic species or other species that may provide desirable organoleptic or other qualities to the composition, e.g. Lactococcus lactis.

Accordingly in one embodiment the present invention provides an inoculum mixture comprising a Streptococcus thermophilus CNCM I-5448 inoculum and further comprising at least one inoculum of Lactobacillus bulgaricus and optionally one or more additional inoculum of Lactococcus lactis and/or Bifidobacterium.

### Methods for the Preparation of Fermented Dairy Products

The S. thermophilus strains of the invention are suitable for use in the preparation of fermented dairy products. Accordingly, an aspect of the present invention also relates to the intended use of these strains, in particular of Streptococcus thermophilus CNCM I-5448, for the preparation of a food composition, more particularly for fermenting a food product.

The present invention also provides a process for the preparation of a fermented dairy product comprising inoculating a milk-based composition with a S. thermophilus strain as defined above and fermenting.

Accordingly, in one embodiment the present invention provides a process comprising the following steps:
i) providing a mixture comprising:
   a) milk, and
   b) a S. thermophilus strain or inoculum as defined above,
ii) fermenting said mixture to provide a fermented dairy product.

In an embodiment, the mixture further comprises sucrose. In a preferred embodiment, the mixture comprises at least 1, 2, 3, 4, 5, 10 or 20% w/w of sucrose, preferably 1-15% w/w, more preferably 2-10% w/w. It is particularly preferred that the sucrose content in the fermented dairy product obtained at step ii) is at least 90% or more, preferably at least 91%, 92% or 93% of the sucrose content in the mixture of step i). It is preferred that said sucrose content is measured after 7 days storage at 4 °C.

Preferably, fermented dairy products are prepared using milk that has been subjected to heat treatment at least equivalent to pasteurization. Preferably, said heat treatment is carried out prior to the preparation of the composition.

Typically, milk is pasteurized by means of the following successive steps:
1) standardization of fatty substances of the raw material so as to obtain a standardized substance,
2) enrichment with dried matter of the standardized substance obtained in the preceding stage, so as to obtain an enriched substance,
3) preheating of the enriched substance obtained in the preceding stage, so as to obtain a starting substance,
4) pasteurization and holding of the starting substance obtained in the preceding stage, so as to obtain a pasteurized and held substance,
5) an optional stage of homogenization of the pasteurized and held substance obtained in the preceding stage, so as to obtain a pasteurized, held and optionally homogenized substance,
6) initial cooling of the pasteurized, held and optionally homogenized substance obtained in the preceding stage, so as to obtain a pasteurized starting substance that has been held, optionally homogenized, and cooled down.

As used herein “standardization of fatty substances” is taken to mean a stage of bringing the quantity of fats present in the starting substance to a pre-determined level. Enrichment with dried matter involves the addition of proteins and fatty substance in order to modify curd firmness.

As used herein “holding” is taken to mean a rapid heating and maintenance of temperature of the milk and makes it possible to destroy the vegetative microbial flora, including pathogenic forms. Its typical duration is from 4 to 10 minutes, in particular from 5 to 8 minutes, and in particular approximately 6 minutes.

As used herein “homogenization” is taken to mean the dispersion of the fatty substances in the milk-type substance into small fat globules. The homogenization is carried out for example at a pressure of 100 to 280 bars, in particular 100 to 250 bars, in particular 100 to 200 bars, in particular approximately 200 bars. This homogenization stage is purely optional. It is in particular absent from the production process of products with 0% fatty substances.

Typically a fermented dairy product is prepared by culture of milks at a suitable temperature with suitable microorganisms to provide a reduction in pH, preferably to a pH equal to or lower than 5, preferably between about 3 and 4.7; more preferably between about 3.5 and about 4.7. The pH can be adjusted by controlling the fermentation by the microorganism and stopping it when appropriate, for example by cooling.

According to an embodiment of the process for the preparation of a fermented dairy product as defined above, the mixture comprising milk and a S. thermophilus of the invention further comprises at least one, two, three or more strains of Bifidobacterium and/or lactic acid bacteria. The selection of suitable Bifidobacterium strains is within the scope of the skilled person and is typically a probiotic lactic acid bacteria. Examples of Bifidobacterium that can be used include but are not limited to Bifidobacterium animalis (for example Bifidobacterium animalis subsp. animalis or Bifidobacterium animalis subsp. lactis); Bifidobacterium longum; Bifidobacterium breve; Bifidobacterium bifidum.

The selection of suitable lactic acid bacteria strains is within the scope of the skilled person and is typically a thermophillic lactic acid bacteria. Examples of lactic acid bacteria that can be used include but are not limited to Lactobacilli (for example Lactobacillus acidophilus, Lactobacillus buchneri, Lactobacillus delbruckeii, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris). Typically a mixture or association of a plurality of species of lactic acid bacteria may be used, typically a mixture or association of Lactobacillus and Streptococcus. For the preparation of yogurt this typically includes Lactobacillus bulgaricus (also referred to as Lactobacillus delbrueckii subsp. bulgaricus) and Streptococcus thermophilus, optionally with additional microorganisms such as but not limited to probiotic species or other species that may provide desirable organoleptic or other qualities to the composition, e.g. Lactococcus lactis.

Accordingly, in one embodiment the mixture further comprises at least one strain of Lactobacillus bulgaricus and optionally one or more strains of Lactococcus lactis and/or Bifidobacterium.

Suitable temperatures for milk fermentation are typically about 36 °C to about 44 °C and the temperature is maintained for an incubation time sufficient to provide the desired reduction in pH. For the preparation of a fermented dairy product the temperature at the start of fermentation is typically about 36 °C to about 43 °C, in particular about 37 °C to about 40 °C, the temperature at the end of fermentation is typically about 37 °C to about 44 °C, in particular about 38 °C to about 41 °C. The fermentation time is typically about 6 to about 11 hours.

Subsequent to the fermentation the fermented dairy product is cooled. Optionally a stage of intermediate cooling of the fermented milk may be performed to provide a pre-cooled fermented milk having a temperature of between about 22 °C and about 4 °C. Typically the intermediate cooling time is about 1 hour to about 4 hours, in particular about 1 hour 30 minutes to about 2 hours. The pre-cooled fermented milk is typically stored for up to 40 hours or less.

Preferably, a stage of final cooling of the fermented milk is performed such that the temperature at the start of the final cooling is less than about 22 °C and the temperature at the end of the final cooling is about 4 °C to about 10 °C. The cooled product may then be stored, transported and/or distributed at a temperature from about 1 °C to about 10 °C for at least about 30 days, at least about 60 days or at least about 90 days.

It is particularly preferred that the amount of sucrose in the fermented dairy product is at least 90% or more, preferably at least 91%, 92% or 93% of the sucrose content in the product mixture prior to fermentation. It is preferred that said sucrose is measured after 7 days storage at 4 °C. According to a further embodiment, the process for the preparation of a fermented dairy product as defined above optionally comprises a stage of stirring at a pressure of at least 20 bars, or performing a dynamic smoothing, to obtain a composition having the desired viscosity, typically a viscosity of up to 20 mPa·s. Stirring or dynamic smoothing operations provide some shear to composition that typically allow a viscosity drop. Such operations are known by the one skilled in the art, and can be operated with conventional appropriate equipment. This stage is typically performed at cold temperature, for example at a temperature of form 1 °C to 20 °C. Without intending to be bound to any theory, it is believed that applying some shear at cold temperature, typically by stirring at high pressure or by performing a dynamic smoothing, can lead to a fluid gel formation within the composition, that provides improved stability even at a low viscosity of up to 20 mPa·s.

Alternatively, according to a further embodiment, the process for the preparation of a fermented dairy product as defined above optionally comprises a stage of acid whey removal to provide a “strained fermented dairy composition”. In this step, an acid whey composition is separated from the curd resulting from the protein coagulation due to acidification during fermentation. Thus one obtains:
a fermented dairy product, typically comprising the proteins coagulum, referred to as a strained fermented dairy composition, and
an acid whey by-product

Such separation steps are known by the one skilled in art, for example in processes of making “greek yogurts”. The separation can for example be carried out by reverse osmosis, ultrafiltration, or centrifugal separation. The separation step can be performed for example at a temperature of from 30 °C to 45 °C.

According to a further embodiment, the process for the preparation of a fermented dairy product as defined above optionally comprises a stage of addition of an intermediate preparation as described above prior or subsequent to fermentation, said intermediate preparation typically comprising a preparation of fruits and/or cereals and/or additives such as flavorings and/or colourings.

The invention will be further illustrated by the following non-limiting Figures and Examples.

### DESCRIPTION OF THE FIGURES

FIG. 1. Growth of wild type strain and Gal+ clones G9 to G16 in M17m with lactose as a sole source of carbon.

FIG. 2. Growth of wild type strain and Gal+ clones G9 to G16 in M17m with galactose as a sole source of carbon.

FIG. 3. Fermentation of semi-skimmed UHT milk by the wild type strain or by the sucrose deficient clones 1-13.

FIG. 4. Growth of wild type strain and sucrose deficient clones 1-13 in M17m with lactose as a sole source of carbon.

FIG. 5. Growth of wild type strain and sucrose deficient clones 1-13 in M17m with sucrose as a sole source of carbon.

FIG. 6. Fermentation of semi-skimmed UHT milk by the wild type strain, clone 11 or clone 11R15 (CNCM I-5448).

FIG. 7. Comparison of the genomes of the wild type strain and of clone G15. (FIG. 7A) Alignment of the lacS nucleotide sequences. (FIG. 7B) Alignment of the LacS amino acid sequences. (FIG. 7C) Alignment of the intergenic region between galR and galK genes. A point mutation is indicated by a box. Cre site in the galK promoter is underlined.

FIG. 8. Comparison of the genomes of clone G15 and of clone 11. (FIG. 8A) Alignment of the scrA nucleotide sequences. (FIG. 8B) Alignment of the ScrA amino acid sequences. A point mutation is indicated by a box.

FIG. 9. Comparison of the genomes of clone 11 and of clone 11R15 (CNCM I-5448). (FIG. 9A) Alignment of the galR nucleotide sequences. (FIG. 9B) Alignment of the GalR amino acid sequences. A point mutation is indicated by a box.

### EXAMPLES

The Inventors aimed to identify a sucrose negative (Suc−) S. thermophilus strain suitable for the preparation of fermented dairy products, such as yogurt said strain being sucrose negative (Suc−) to ensure improved sweetness in sugar containing products with minimal post-acidification.

Additionally it was required that said strain have texturizing properties (i.e. producing EPS) and furthermore it was preferred that the strain was fructose negative (Fru−) to ensure that the improved sweetness and reduced post-acidification in products containing fruit.

Accordingly, the Inventors carried out a screening program of 655 S. thermophilus strains from the Applicant's Danone Culture Collection. Each strain was analysed using the API 50CH test after 48 hours of incubation at 37 °C with MRS API. This made it possible to determine the fermentation profile for galactose, sucrose and fructose.

Of all the strains tested 654 were Suc+, 13 were Gal+, 84 were Fru+. All Gal+strains (13) were also Fru+. Of all the strains tested no Suc− Gal+ was identified. The sole Suc− strain identified was Glu+, Fru+, Lac+, Gal−.

I. Selection of a S. thermophilus Gal+ Variant The wild type strain (Wt) shows the phenotype Lac+ Glu+ Suc+ Gal− Fru− and efficiently carries out the fermentation of lactose with high texturizing properties. The wild type strain was successively subcultured in modified M17 medium (M17m, the concentration of beta-glycerophosphate is 9 g/l instead of 19 g/l in M17) in the presence of galactose as the sole carbon source. After 12 subcultures, some clones were isolated on dishes containing M17m galactose. Eight clones (G9 to G16) were isolated and cultured in M17m galactose at 5 g/l.

The growth of the eight selected clones was tested and monitored using a Bioscreen C at a wavelength of 600 nm (Bioscreen C is a spectrophotometer allowing the monitoring of growth by measuring the optical density at 600 nm in microplates) in two different media. Growth was tested in the presence (i) of lactose as the sole source of carbon or (ii) of galactose as the sole source of carbon. The precultures were carried out in M17m gal for the colonies and in M17m lac for the wild type strain. The inoculation rate was 1% from the precultures.

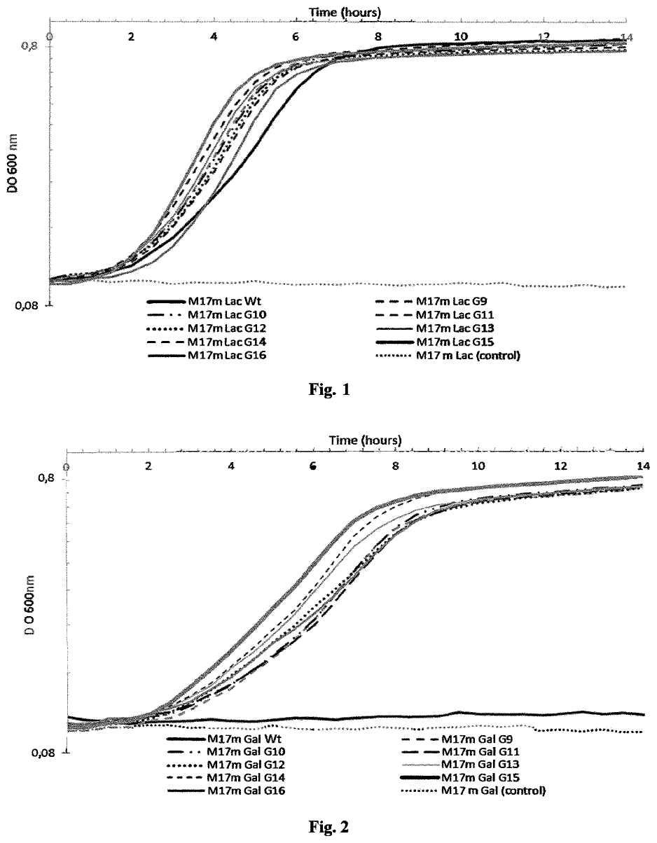

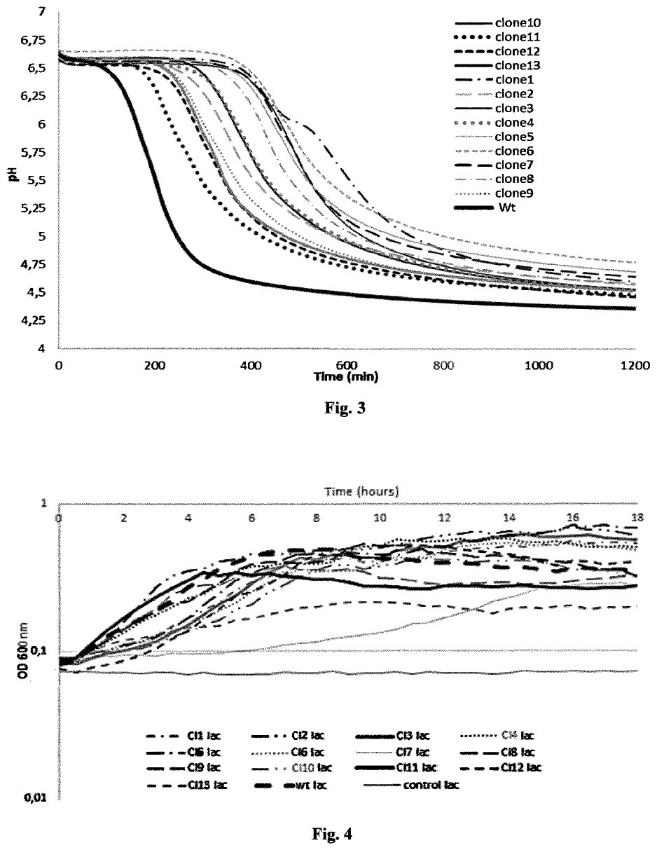

The wild type strain and the eight isolated clones efficiently grow in the medium containing lactose (FIG. 1), whereas, in the medium containing galactose, only the eight isolated clones are able to grow (FIG. 2).

These results show the isolation of Gal+variants from the Gal− wild type strain.

Clone G15 was selected because it showed the best growth in both galactose and lactose.

II. Mutagenesis of the S. thermophilus Gal+ Variant

Ultraviolet mutagenesis was performed on the Gal+ clone G15 derived from the wild type strain. For the mutagenesis, a dose of UV giving approximately 2% survival was used.

Four thousand clones obtained from mutagenesis were tested for their capacity to acidify the M17m medium in the presence of galactose at 5 g/l as a carbon source and to acidify the M17m medium in the presence of sucrose at 5 g/l as a source of carbon. Purple bromocresol was used as a pH indicator, which turns yellow in acidic medium while it is purple in a non-inoculated medium.

Thirteen out of 4000 tested clones showed a reduced ability to acidify the medium in the presence of sucrose while maintaining a good acidification in the presence of galactose as a carbon source.

III. Characterization of the S. thermophilus Gal+ Mutants with a Deficiency in Sucrose Metabolism

13 clones were tested in semi-skimmed UHT milk supplemented with yeast extract at 2 g/l. The milk enriched with yeast extract was inoculated at 1% from a pre-culture made in semi-skimmed UHT milk enriched with yeast extract at 2 g/l. The cultures were incubated at 38 °C and the fermentation was stopped at pH 4.7 by cooling to 4 °C.

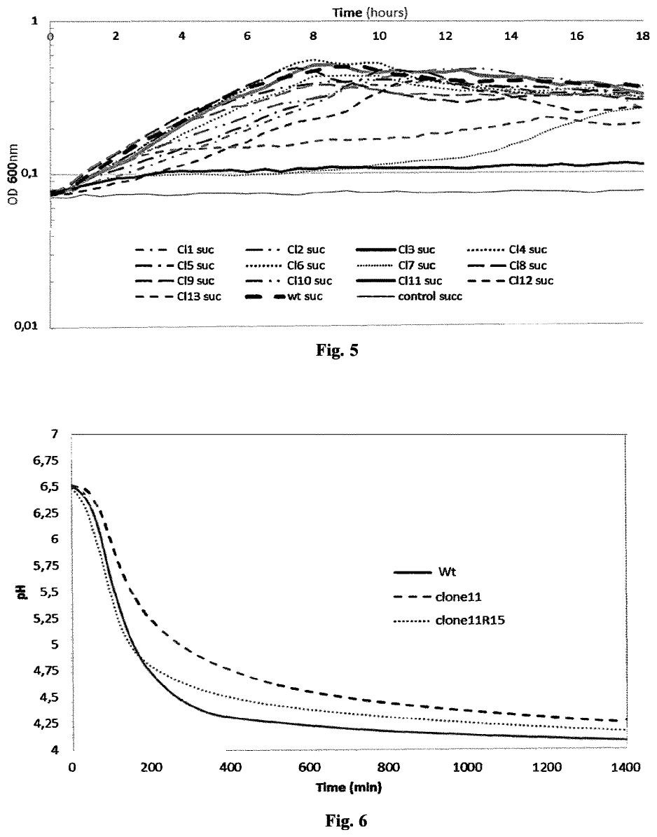

The fermentation was monitored by the iCinac system which makes it possible to obtain the pH values continuously. All clones showed a reduced capacity to acidify the milk (FIG. 3).

The ability of the clones to grow in a medium containing sucrose as a carbon source was also evaluated by monitoring the optical density at 600 nm with Bioscreen C.

Growth monitoring was carried out in the presence (i) of lactose as the sole source of carbon or (ii) of sucrose as the sole source of carbon. The precultures were carried out in M17m gal for the colonies and in M17m Lac for the wild type strain. The inoculation rate was 1% from the precultures.

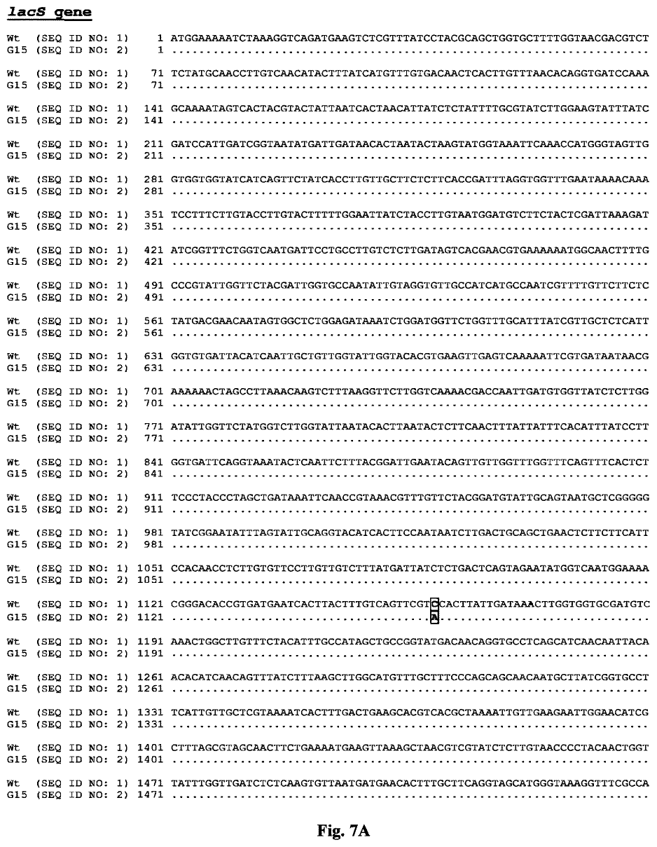

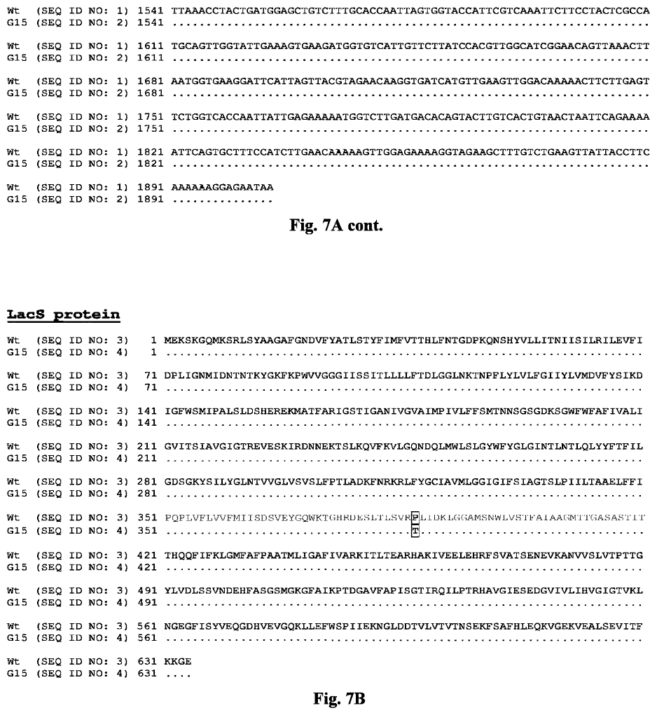

In the presence of lactose, all clones were able to grow (FIG. 4). In the presence of sucrose, most of the clones showed a growth that was reduced as compared to the growth of wild type strain, but only clone 11 showed an inability to grow in the presence of sucrose (FIG. 5).

These results show the successful isolation of a Suc− clone from the Gal+variant.

### IV. Improvement of the Growth of the Gal+ Suc− Strain in Milk.

Clone 11 showed a clear Suc− phenotype, but also showed a reduction of its fermentative efficacy in milk. In order to restore an efficacy similar to the one of the wild type strain, 15 successive subcultures in semi-skimmed UHT milk supplemented with yeast extract at 2 g/l were carried out. Clone 11R15 was obtained and subsequently deposited as strain CNCM I-5448, as described above. The fermentation with the wild type strain, clone 11 or clone 11R15 in UHT semi-skimmed milk with 2 g/l yeast extract was measured by the iCinac system.

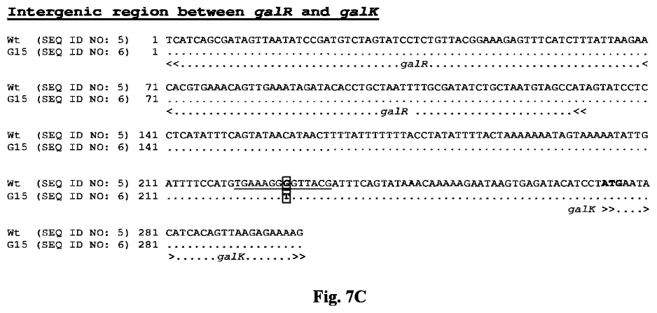

Clone 11R15 (CNCM I-5448) showed a capacity to acidify milk better than that of clone 11 and similar to that of the wild type strain (FIG. 6).

These results show the isolation of a Suc− variant with the same capacity to ferment milk as compared to the wild type strain.

### V. Characterization of the Clone 11R15 (CNCM I-5448)

### V.1. Dosage of Sucrose

Mixtures were prepared using UHT semi-skimmed milk with 2 g/l yeast extract and 55 g/l sucrose and inoculated with clone 11R15 or with the wild type strain, fermentation was carried out at 38 °C and stopped when the pH was 4.7 by rapid cooling.

As a control an unfermented acidic (pH 4.7) milk product (no strains) was prepared using UHT semi-skimmed milk with 2 g/l yeast extract and 55 g/l sucrose and glucono delta-lactone (GDL) 1.5% w/w as an acidifying agent. Said control product was heated to 38 °C for 4 hours, to ensure comparability to test products.

Products were stored for 7 days at 4 °C, then the products were centrifuged at 5000 g for 5 minutes at 4 °C. The determination of sucrose in the supernatants was assayed on 3 samples in 2 experiments using a Glucose-Fructose-Sucrose assay kit from Biosentec. The results are given in Table 1.

TABLE 1
Level of sucrose in the products after fermentation with wild type
strain or with clone 11R15 (CNCM I-5448).

| | First | Standard | Second | Standard |
|---|---|---|---|---|
| | experiment | deviation | experiment | deviation |
| Wt | 47.7 g/L | 1.77 | 44.1 g/L | 2.36 |
| clone 11R15 | 52.9 g/L | 2 | 53 g/L | 2.15 |
| (CNCM I-5448) | | | | |
| GDL 1.5% | 55.46 g/L | 1.22 | 52.16 g/L | 1.09 |
| Control | | | | |

In the first experiment, the product fermented with the wild type strain has lost 7.3±1.8 g whereas the product fermented with the clone 11R15 has lost only 2.1±1.8 g. In the second experiment, the product fermented with the wild type strain has lost 10.9±2 g whereas the product fermented with the clone 11R15 has lost only 2±2.1 g.

These results show that clone 11R15 (CNCM I-5448) is able to achieve fermentation while significantly maintaining the level of sucrose in the ingredients, contrary to the wild type strain. The slight reduction in sucrose during fermentation can reasonably be attributed to the inherent degradation of sucrose in the acidic pH, as it is comparable to that of the GDL control in the second experiment.

### V.2. Viscosity

Mixtures were prepared using UHT semi-skimmed milk with 2 g/l yeast extract and 55 g/l sucrose and inoculated with clone 11R15, with clone 11 or with wild type strain. fermentation was carried out at 38 °C and stopped when the pH was 4.7 by rapid cooling. Products were stored for 7 days at 4 °C, then the viscosity was measured by shear stress measurement.

This method consists of determining the viscosity of products, after manual stirring and incubation for 30 minutes at 4 °C. Three measurements were carried out at 4 °C on three pots of milk fermented by the same strain and under the same conditions. The apparatus used for this analysis was a refrigerated MCR301 rheometer equipped with a 27 mm concentric cylinder system. This rotating system makes it possible to observe a destructurisation of the product as a function of a linear shear gradient, i.e. a stress at a given gradient. The results are obtained in the form of a continuous flow curve, rising and falling ramp between 0 and 20 s-1. The product undergoes an increasing shear gradient from 0 to 20 s-1 for 1 minute. This phase corresponds to the rising ramp. Then, it undergoes a decreasing shear gradient from 20 to 0 s-1 for 1 minute, corresponding to the downward ramp.

Each downward curve is then modeled according to the Casson model (equation I).

$$\sqrt{τ} = \sqrt{{τ}_{0}} + \sqrt{η \times D} \qquad ( 1 )$$

τ: Stress (Pa)
τ₀: Yield point of the product (Pa)
η: Viscosity of the product (Pa·s)
D: Shear gradient (s-1)
   Viscosity of the products after 7 days of storage at 4 °C are given in Table 2.

TABLE 2
Viscosity the products fermented with wild type strain,
with clone 11 or with clone 11R15.

| | | Standard |
|---|---|---|
| | Viscosity | deviation |
| Wt | 1116 mPa · s | 76 |
| clone 11 | 557 mPa · s | 36 |
| clone 11R15 | 1143 mPa · s | 42 |

These results show that clone 11R15 (CNCM I-5448) has similar texturizing properties as compared to the wild type strain.

### V.3. API (Analytical Profile Index) Test

The fermentation profile of the clone 11R15 (CNCM I-5448) was obtained by carrying out the API 50CH test after 48 hours of incubation at 37 °C with MRS API. This made it possible to determine the fermentation profile for galactose, sucrose and fructose.

Clone 11R15 (CNCM I-5448) is galactose positive (Gal+), sucrose negative (Suc−), fructose negative (Fru−), lactose positive (Lac+), glucose positive (Glu+) as determined using said test and furthermore is highly fermentative and texturizing (significantly EPS producing).

VI. Genomic Comparisons of the S. thermophilus Variants

The complete genomes of the S. thermophilus wild strain and of its variants, i.e. clone G15, clone 11 and clone 11R15, were sequenced by NGS using Illumina NovaSeq6000 system.

The genomic sequences of all variants were compared to each other in order to identify the mutations associated to specific phenotypes which appeared step by step to the isolation of clone 11R15 (CNCM I-5448).

### VI1. Gal+ Phenotype

The comparison of the genome of the wild type strain with the genome of clone G15 showed that a mutation in the lacS gene and a mutation in the intergenic region between ga/R and galK were associated with the Gal+ phenotype.

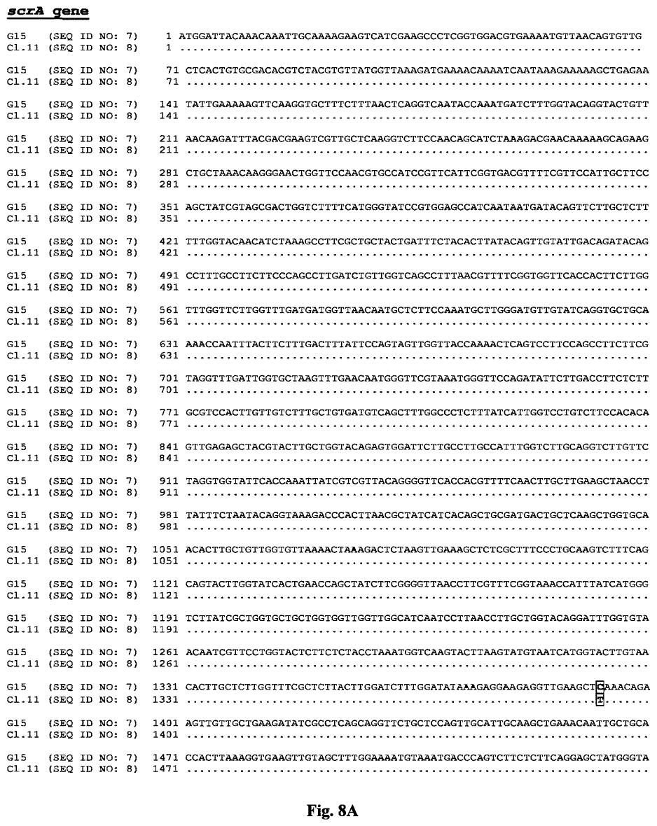

In particular, the Inventors noted a substitution C/A in the lacS gene in position 1159 of SEQ ID NO: 1 (FIG. 7A), which results in a replacement of a Proline by a Threonine in position 387 of the LacS protein of SEQ ID NO: 3 (FIG. 7B), and a substitution G/T in galK promoter in position 228 of SEQ ID NO: 5 (FIG. 7C).

### VI.2. Suc− Phenotype

The comparison of the genome of clone G15 with the genome of clone 11 showed that a non-sense mutation in the scrA gene was associated with the Suc− phenotype.

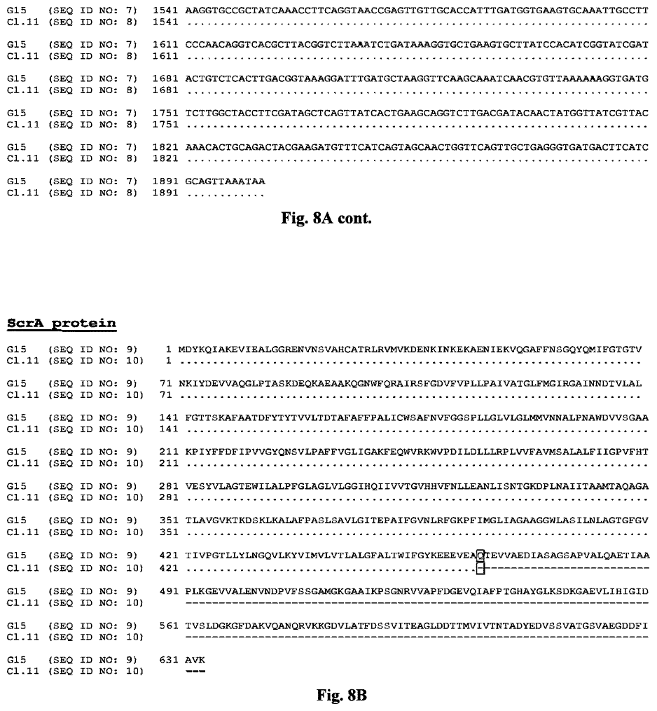

In particular, the Inventors noted a non-sense substitution C/T in the scrA gene in position 1393 of SEQ ID NO: 7 (FIG. 8A), which results in a stop in the ScrA protein in position 465 of SEQ ID NO: 9 (FIG. 8B).

### VI.2. Rapid Growth in Milk

The comparison of the genome of clone 11 with the genome of clone 11R15 (CNCM I-5448) showed that a mutation in the galR gene was associated with the improvement of the growth of the strain in milk.

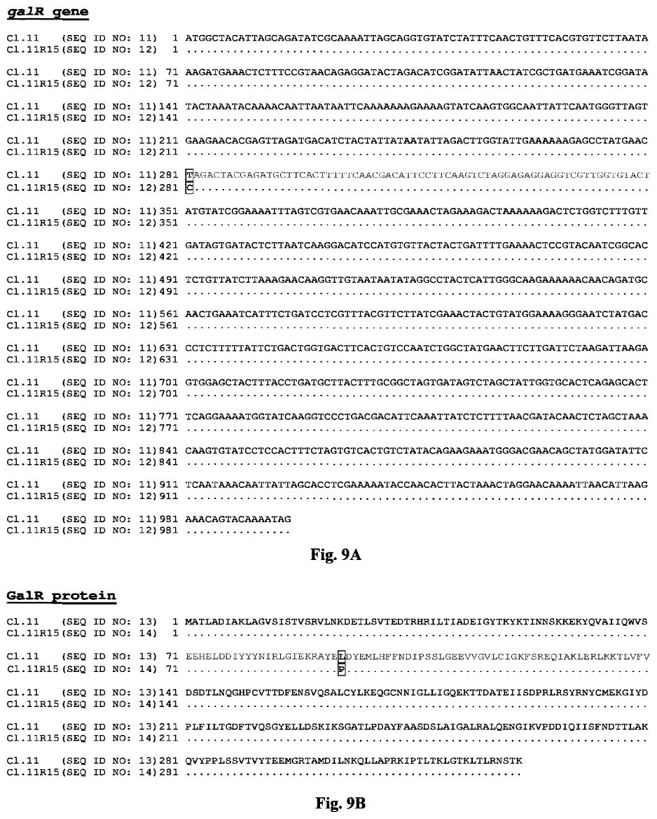

In particular, the Inventors noted a substitution T/C in the ga/R gene in position 281 of SEQ ID NO: 11 (FIG. 9A), which results in a replacement of a Leucine by a Proline in the GalR protein in position 94 of SEQ ID NO: 13 (FIG. 9B).

The mutations are summarized in order of appearance in Table 3.

TABLE 3
Summary of the step-by-step mutations between
S. thermophilus variants.

| | | | | Clone 11R15 |
|---|---|---|---|---|
| | | | | (CNCM I-5448) |
| | | | Clone | Gal+ |
| | Wild | Clone | 11 | Suc− |
| | type | G15 | Gal+ | Rapid growth |
| Mutation | strain | Gal+ | Suc− | in milk |
| lacS | No | yes | yes | yes |
| Substitution C/A (P→T) | | | | |
| Intergenic region between | No | yes | yes | yes |
| galR and galK | | | | |
| Substitution G/T | | | | |
| in galK promoter (cre site) | | | | |
| scrA | No | No | yes | yes |
| Substitution C/T | | | | |
| (Non-sense mutation) | | | | |
| galR | No | No | No | yes |
| Substitution T/C (L→P) | | | | |

1. A mutant Streptococcus thermophilus strain that it is galactose positive and sucrose negative.

2. The mutant strain of claim 1, a wherein the strain is a texturizing strain.

3. The mutant strain of claim 1, wherein the strain is glucose positive.

4. A S. thermophilus strain deposited at the CNCM under reference number CNCM I-5448.

5. A composition comprising the mutant strain of claim 1.

6. The composition according to claim 5, wherein the composition comprises at least 10⁵ CFU/g of said mutant strain.

7. The composition of claim 5, wherein said composition is a food product.

8. The composition of claim 7, wherein said food product is a dairy product or a dairy alternative.

9. A method for preparing a fermented food product comprising:
i) providing a mixture comprising:
a) milk, and
b) at least one S. thermophilus strain of claim 1, and
ii) fermenting said mixture to provide a fermented food product.

10. The composition of claim 5, wherein the composition has a viscosity of at least 800 mPa·s.

11. The composition of claim 5, wherein the composition has a pH of 4.7 or lower.

12. The composition of claim 5, further comprising at least one strain of Bifidobacterium or lactic acid bacteria.

13. (canceled)

14. A method for obtaining a S. thermophilus strain according to claim 1, comprising:
i) providing a first S. thermophilus strain,
ii) selecting at least one galactose positive clone thereof, and
iii) mutating said selected clone(s) selected in ii), and selecting at least one sucrose negative variant thereof.

15. The method of claim 14, further comprising:
iv) selecting a sucrose negative variant that is the most fermentative in a milk based medium and/or subculturing a sucrose negative variant in a milk based medium.


SEQUENCE LISTING (15 sequences)

SEQ ID NO: 1 - DNA, 1905 bp, Streptococcus thermophilus
  source 1..1905: mol_type = other DNA; note = Streptococcus thermophilus wild type
atggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttgtaatggatgtcttctactcgattaaagatatcggtttctggtcaatgattcctgccttgtctcttgatagtcacgaacgtgaaaaaatggcaacttttgcccgtattggttctacgattggtgccaatattgtaggtgttgccatcatgccaatcgttttgttcttctctatgacgaacaatagtggctctggagataaatctggatggttctggtttgcatttatcgttgctctcattggtgtgattacatcaattgctgttggtattggtacacgtgaagttgagtcaaaaattcgtgataataacgaaaaaactagccttaaacaagtctttaaggttcttggtcaaaacgaccaattgatgtggttatctcttggatattggttctatggtcttggtattaatacacttaatactcttcaactttattatttcacatttatccttggtgattcaggtaaatactcaattctttacggattgaatacagttgttggtttggtttcagtttcactcttccctaccctagctgataaattcaaccgtaaacgtttgttctacggatgtattgcagtaatgctcgggggtatcggaatatttagtattgcaggtacatcacttccaataatcttgactgcagctgaactcttcttcattccacaacctcttgtgttccttgttgtctttatgattatctctgactcagtagaatatggtcaatggaaaacgggacaccgtgatgaatcacttactttgtcagttcgtccacttattgataaacttggtggtgcgatgtcaaactggcttgtttctacatttgccatagctgccggtatgacaacaggtgcctcagcatcaacaattacaacacatcaacagtttatctttaagcttggcatgtttgctttcccagcagcaacaatgcttatcggtgccttcattgttgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcatgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataa

SEQ ID NO: 2 - DNA, 1905 bp, Streptococcus thermophilus
  source 1..1905: mol_type = other DNA; note = Streptococcus thermophilus G15
atggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttgtaatggatgtcttctactcgattaaagatatcggtttctggtcaatgattcctgccttgtctcttgatagtcacgaacgtgaaaaaatggcaacttttgcccgtattggttctacgattggtgccaatattgtaggtgttgccatcatgccaatcgttttgttcttctctatgacgaacaatagtggctctggagataaatctggatggttctggtttgcatttatcgttgctctcattggtgtgattacatcaattgctgttggtattggtacacgtgaagttgagtcaaaaattcgtgataataacgaaaaaactagccttaaacaagtctttaaggttcttggtcaaaacgaccaattgatgtggttatctcttggatattggttctatggtcttggtattaatacacttaatactcttcaactttattatttcacatttatccttggtgattcaggtaaatactcaattctttacggattgaatacagttgttggtttggtttcagtttcactcttccctaccctagctgataaattcaaccgtaaacgtttgttctacggatgtattgcagtaatgctcgggggtatcggaatatttagtattgcaggtacatcacttccaataatcttgactgcagctgaactcttcttcattccacaacctcttgtgttccttgttgtctttatgattatctctgactcagtagaatatggtcaatggaaaacgggacaccgtgatgaatcacttactttgtcagttcgtacacttattgataaacttggtggtgcgatgtcaaactggcttgtttctacatttgccatagctgccggtatgacaacaggtgcctcagcatcaacaattacaacacatcaacagtttatctttaagcttggcatgtttgctttcccagcagcaacaatgcttatcggtgccttcattgttgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcatgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataa

SEQ ID NO: 3 - protein, 634 aa, Streptococcus thermophilus
  source 1..634: note = Streptococcus thermophilus wild type
MEKSKGQMKSRLSYAAGAFGNDVFYATLSTYFIMFVTTHLFNTGDPKQNSHYVLLITNIISILRILEVFIDPLIGNMIDNTNTKYGKFKPWVVGGGIISSITLLLLFTDLGGLNKTNPFLYLVLFGIIYLVMDVFYSIKDIGFWSMIPALSLDSHEREKMATFARIGSTIGANIVGVAIMPIVLFFSMTNNSGSGDKSGWFWFAFIVALIGVITSIAVGIGTREVESKIRDNNEKTSLKQVFKVLGQNDQLMWLSLGYWFYGLGINTLNTLQLYYFTFILGDSGKYSILYGLNTVVGLVSVSLFPTLADKFNRKRLFYGCIAVMLGGIGIFSIAGTSLPIILTAAELFFIPQPLVFLVVFMIISDSVEYGQWKTGHRDESLTLSVRPLIDKLGGAMSNWLVSTFAIAAGMTTGASASTITTHQQFIFKLGMFAFPAATMLIGAFIVARKITLTEARHAKIVEELEHRFSVATSENEVKANVVSLVTPTTGYLVDLSSVNDEHFASGSMGKGFAIKPTDGAVFAPISGTIRQILPTRHAVGIESEDGVIVLIHVGIGTVKLNGEGFISYVEQGDHVEVGQKLLEFWSPIIEKNGLDDTVLVTVTNSEKFSAFHLEQKVGEKVEALSEVITFKKGE

SEQ ID NO: 4 - protein, 634 aa, Streptococcus thermophilus
  source 1..634: note = Streptococcus thermophilus G15
MEKSKGQMKSRLSYAAGAFGNDVFYATLSTYFIMFVTTHLFNTGDPKQNSHYVLLITNIISILRILEVFIDPLIGNMIDNTNTKYGKFKPWVVGGGIISSITLLLLFTDLGGLNKTNPFLYLVLFGIIYLVMDVFYSIKDIGFWSMIPALSLDSHEREKMATFARIGSTIGANIVGVAIMPIVLFFSMTNNSGSGDKSGWFWFAFIVALIGVITSIAVGIGTREVESKIRDNNEKTSLKQVFKVLGQNDQLMWLSLGYWFYGLGINTLNTLQLYYFTFILGDSGKYSILYGLNTVVGLVSVSLFPTLADKFNRKRLFYGCIAVMLGGIGIFSIAGTSLPIILTAAELFFIPQPLVFLVVFMIISDSVEYGQWKTGHRDESLTLSVRTLIDKLGGAMSNWLVSTFAIAAGMTTGASASTITTHQQFIFKLGMFAFPAATMLIGAFIVARKITLTEARHAKIVEELEHRFSVATSENEVKANVVSLVTPTTGYLVDLSSVNDEHFASGSMGKGFAIKPTDGAVFAPISGTIRQILPTRHAVGIESEDGVIVLIHVGIGTVKLNGEGFISYVEQGDHVEVGQKLLEFWSPIIEKNGLDDTVLVTVTNSEKFSAFHLEQKVGEKVEALSEVITFKKGE

SEQ ID NO: 5 - DNA, 300 bp, Streptococcus thermophilus
  source 1..300: mol_type = other DNA; note = Streptococcus thermophilus wild type
tcatcagcgatagttaatatccgatgtctagtatcctctgttacggaaagagtttcatctttattaagaacacgtgaaacagttgaaatagatacacctgctaattttgcgatatctgctaatgtagccatagtatcctcctcatatttcagtataacataacttttatttttttacctatattttactaaaaaaatagtaaaaatattgattttccatgtgaaaggggttacgatttcagtataaacaaaaagaataagtgagatacatcctatgaatacatcacagttaagagaaaag

SEQ ID NO: 6 - DNA, 300 bp, Streptococcus thermophilus
  source 1..300: mol_type = other DNA; note = Streptococcus thermophilus G15
tcatcagcgatagttaatatccgatgtctagtatcctctgttacggaaagagtttcatctttattaagaacacgtgaaacagttgaaatagatacacctgctaattttgcgatatctgctaatgtagccatagtatcctcctcatatttcagtataacataacttttatttttttacctatattttactaaaaaaatagtaaaaatattgattttccatgtgaaaggtgttacgatttcagtataaacaaaaagaataagtgagatacatcctatgaatacatcacagttaagagaaaag

SEQ ID NO: 7 - DNA, 1902 bp, Streptococcus thermophilus
  source 1..1902: mol_type = other DNA; note = Streptococcus thermophilus G15
atggattacaaacaaattgcaaaagaagtcatcgaagccctcggtggacgtgaaaatgttaacagtgttgctcactgtgcgacacgtctacgtgttatggttaaagatgaaaacaaaatcaataaagaaaaagctgagaatattgaaaaagttcaaggtgctttctttaactcaggtcaataccaaatgatctttggtacaggtactgttaacaagatttacgacgaagtcgttgctcaaggtcttccaacagcatctaaagacgaacaaaaagcagaagctgctaaacaagggaactggttccaacgtgccatccgttcattcggtgacgttttcgttccattgcttccagctatcgtagcgactggtcttttcatgggtatccgtggagccatcaataatgatacagttcttgctctttttggtacaacatctaaagccttcgctgctactgatttctacacttatacagttgtattgacagatacagcctttgccttcttcccagccttgatctgttggtcagcctttaacgttttcggtggttcaccacttcttggtttggttcttggtttgatgatggttaacaatgctcttccaaatgcttgggatgttgtatcaggtgctgcaaaaccaatttacttctttgactttattccagtagttggttaccaaaactcagtccttccagccttcttcgtaggtttgattggtgctaagtttgaacaatgggttcgtaaatgggttccagatattcttgaccttctcttgcgtccacttgttgtctttgctgtgatgtcagctttggccctctttatcattggtcctgtcttccacacagttgagagctacgtacttgctggtacagagtggattcttgccttgccatttggtcttgcaggtcttgttctaggtggtattcaccaaattatcgtcgttacaggggttcaccacgttttcaacttgcttgaagctaaccttatttctaatacaggtaaagacccacttaacgctatcatcacagctgcgatgactgctcaagctggtgcaacacttgctgttggtgttaaaactaaagactctaagttgaaagctctcgctttccctgcaagtctttcagcagtacttggtatcactgaaccagctatcttcggggttaaccttcgtttcggtaaaccatttatcatgggtcttatcgctggtgctgctggtggttggttggcatcaatccttaaccttgctggtacaggatttggtgtaacaatcgttcctggtactcttctctacctaaatggtcaagtacttaagtatgtaatcatggtacttgtaacacttgctcttggtttcgctcttacttggatctttggatataaagaggaagaggttgaagctcaaacagaagttgttgctgaagatatcgcctcagcaggttctgctccagttgcattgcaagctgaaacaattgctgcaccacttaaaggtgaagttgtagctttggaaaatgtaaatgacccagtcttctcttcaggagctatgggtaaaggtgccgctatcaaaccttcaggtaaccgagttgttgcaccatttgatggtgaagtgcaaattgccttcccaacaggtcacgcttacggtcttaaatctgataaaggtgctgaagtgcttatccacatcggtatcgatactgtctcacttgacggtaaaggatttgatgctaaggttcaagcaaatcaacgtgttaaaaaaggtgatgtcttggctaccttcgatagctcagttatcactgaagcaggtcttgacgatacaactatggttatcgttacaaacactgcagactacgaagatgtttcatcagtagcaactggttcagttgctgagggtgatgacttcatcgcagttaaataa

SEQ ID NO: 8 - DNA, 1902 bp, Streptococcus thermophilus
  source 1..1902: mol_type = other DNA; note = Streptococcus thermophilus clone 11
atggattacaaacaaattgcaaaagaagtcatcgaagccctcggtggacgtgaaaatgttaacagtgttgctcactgtgcgacacgtctacgtgttatggttaaagatgaaaacaaaatcaataaagaaaaagctgagaatattgaaaaagttcaaggtgctttctttaactcaggtcaataccaaatgatctttggtacaggtactgttaacaagatttacgacgaagtcgttgctcaaggtcttccaacagcatctaaagacgaacaaaaagcagaagctgctaaacaagggaactggttccaacgtgccatccgttcattcggtgacgttttcgttccattgcttccagctatcgtagcgactggtcttttcatgggtatccgtggagccatcaataatgatacagttcttgctctttttggtacaacatctaaagccttcgctgctactgatttctacacttatacagttgtattgacagatacagcctttgccttcttcccagccttgatctgttggtcagcctttaacgttttcggtggttcaccacttcttggtttggttcttggtttgatgatggttaacaatgctcttccaaatgcttgggatgttgtatcaggtgctgcaaaaccaatttacttctttgactttattccagtagttggttaccaaaactcagtccttccagccttcttcgtaggtttgattggtgctaagtttgaacaatgggttcgtaaatgggttccagatattcttgaccttctcttgcgtccacttgttgtctttgctgtgatgtcagctttggccctctttatcattggtcctgtcttccacacagttgagagctacgtacttgctggtacagagtggattcttgccttgccatttggtcttgcaggtcttgttctaggtggtattcaccaaattatcgtcgttacaggggttcaccacgttttcaacttgcttgaagctaaccttatttctaatacaggtaaagacccacttaacgctatcatcacagctgcgatgactgctcaagctggtgcaacacttgctgttggtgttaaaactaaagactctaagttgaaagctctcgctttccctgcaagtctttcagcagtacttggtatcactgaaccagctatcttcggggttaaccttcgtttcggtaaaccatttatcatgggtcttatcgctggtgctgctggtggttggttggcatcaatccttaaccttgctggtacaggatttggtgtaacaatcgttcctggtactcttctctacctaaatggtcaagtacttaagtatgtaatcatggtacttgtaacacttgctcttggtttcgctcttacttggatctttggatataaagaggaagaggttgaagcttaaacagaagttgttgctgaagatatcgcctcagcaggttctgctccagttgcattgcaagctgaaacaattgctgcaccacttaaaggtgaagttgtagctttggaaaatgtaaatgacccagtcttctcttcaggagctatgggtaaaggtgccgctatcaaaccttcaggtaaccgagttgttgcaccatttgatggtgaagtgcaaattgccttcccaacaggtcacgcttacggtcttaaatctgataaaggtgctgaagtgcttatccacatcggtatcgatactgtctcacttgacggtaaaggatttgatgctaaggttcaagcaaatcaacgtgttaaaaaaggtgatgtcttggctaccttcgatagctcagttatcactgaagcaggtcttgacgatacaactatggttatcgttacaaacactgcagactacgaagatgtttcatcagtagcaactggttcagttgctgagggtgatgacttcatcgcagttaaataa

SEQ ID NO: 9 - protein, 633 aa, Streptococcus thermophilus
  source 1..633: note = Streptococcus thermophilus G15
MDYKQIAKEVIEALGGRENVNSVAHCATRLRVMVKDENKINKEKAENIEKVQGAFFNSGQYQMIFGTGTVNKIYDEVVAQGLPTASKDEQKAEAAKQGNWFQRAIRSFGDVFVPLLPAIVATGLFMGIRGAINNDTVLALFGTTSKAFAATDFYTYTVVLTDTAFAFFPALICWSAFNVFGGSPLLGLVLGLMMVNNALPNAWDVVSGAAKPIYFFDFIPVVGYQNSVLPAFFVGLIGAKFEQWVRKWVPDILDLLLRPLVVFAVMSALALFIIGPVFHTVESYVLAGTEWILALPFGLAGLVLGGIHQIIVVTGVHHVFNLLEANLISNTGKDPLNAIITAAMTAQAGATLAVGVKTKDSKLKALAFPASLSAVLGITEPAIFGVNLRFGKPFIMGLIAGAAGGWLASILNLAGTGFGVTIVPGTLLYLNGQVLKYVIMVLVTLALGFALTWIFGYKEEEVEAQTEVVAEDIASAGSAPVALQAETIAAPLKGEVVALENVNDPVFSSGAMGKGAAIKPSGNRVVAPFDGEVQIAFPTGHAYGLKSDKGAEVLIHIGIDTVSLDGKGFDAKVQANQRVKKGDVLATFDSSVITEAGLDDTTMVIVTNTADYEDVSSVATGSVAEGDDFIAVK

SEQ ID NO: 10 - protein, 464 aa, Streptococcus thermophilus
  source 1..464: note = Streptococcus thermophilus clone 11
MDYKQIAKEVIEALGGRENVNSVAHCATRLRVMVKDENKINKEKAENIEKVQGAFFNSGQYQMIFGTGTVNKIYDEVVAQGLPTASKDEQKAEAAKQGNWFQRAIRSFGDVFVPLLPAIVATGLFMGIRGAINNDTVLALFGTTSKAFAATDFYTYTVVLTDTAFAFFPALICWSAFNVFGGSPLLGLVLGLMMVNNALPNAWDVVSGAAKPIYFFDFIPVVGYQNSVLPAFFVGLIGAKFEQWVRKWVPDILDLLLRPLVVFAVMSALALFIIGPVFHTVESYVLAGTEWILALPFGLAGLVLGGIHQIIVVTGVHHVFNLLEANLISNTGKDPLNAIITAAMTAQAGATLAVGVKTKDSKLKALAFPASLSAVLGITEPAIFGVNLRFGKPFIMGLIAGAAGGWLASILNLAGTGFGVTIVPGTLLYLNGQVLKYVIMVLVTLALGFALTWIFGYKEEEVEA

SEQ ID NO: 11 - DNA, 996 bp, Streptococcus thermophilus
  source 1..996: mol_type = other DNA; note = Streptococcus thermophilus clone 11
atggctacattagcagatatcgcaaaattagcaggtgtatctatttcaactgtttcacgtgttcttaataaagatgaaactctttccgtaacagaggatactagacatcggatattaactatcgctgatgaaatcggatatactaaatacaaaacaattaataattcaaaaaaagaaaagtatcaagtggcaattattcaatgggttagtgaagaacacgagttagatgacatctactattataatattagacttggtattgaaaaaagagcctatgaactagactacgagatgcttcactttttcaacgacattccttcaagtctaggagaggaggtcgttggtgtactatgtatcggaaaatttagtcgtgaacaaattgcgaaactagaaagactaaaaaagactctggtctttgttgatagtgatactcttaatcaaggacatccatgtgttactactgattttgaaaactccgtacaatcggcactctgttatcttaaagaacaaggttgtaataatataggcctactcattgggcaagaaaaaacaacagatgcaactgaaatcatttctgatcctcgtttacgttcttatcgaaactactgtatggaaaagggaatctatgaccctctttttattctgactggtgacttcactgtccaatctggctatgaacttcttgattctaagattaagagtggagctactttacctgatgcttactttgcggctagtgatagtctagctattggtgcactcagagcacttcaggaaaatggtatcaaggtccctgacgacattcaaattatctcttttaacgatacaactctagctaaacaagtgtatcctccactttctagtgtcactgtctatacagaagaaatgggacgaacagctatggatattctcaataaacaattattagcacctcgaaaaataccaacacttactaaactaggaacaaaattaacattaagaaacagtacaaaatag

SEQ ID NO: 12 - DNA, 996 bp, Streptococcus thermophilus
  source 1..996: mol_type = other DNA; note = Streptococcus thermophilus clone 11R15
atggctacattagcagatatcgcaaaattagcaggtgtatctatttcaactgtttcacgtgttcttaataaagatgaaactctttccgtaacagaggatactagacatcggatattaactatcgctgatgaaatcggatatactaaatacaaaacaattaataattcaaaaaaagaaaagtatcaagtggcaattattcaatgggttagtgaagaacacgagttagatgacatctactattataatattagacttggtattgaaaaaagagcctatgaaccagactacgagatgcttcactttttcaacgacattccttcaagtctaggagaggaggtcgttggtgtactatgtatcggaaaatttagtcgtgaacaaattgcgaaactagaaagactaaaaaagactctggtctttgttgatagtgatactcttaatcaaggacatccatgtgttactactgattttgaaaactccgtacaatcggcactctgttatcttaaagaacaaggttgtaataatataggcctactcattgggcaagaaaaaacaacagatgcaactgaaatcatttctgatcctcgtttacgttcttatcgaaactactgtatggaaaagggaatctatgaccctctttttattctgactggtgacttcactgtccaatctggctatgaacttcttgattctaagattaagagtggagctactttacctgatgcttactttgcggctagtgatagtctagctattggtgcactcagagcacttcaggaaaatggtatcaaggtccctgacgacattcaaattatctcttttaacgatacaactctagctaaacaagtgtatcctccactttctagtgtcactgtctatacagaagaaatgggacgaacagctatggatattctcaataaacaattattagcacctcgaaaaataccaacacttactaaactaggaacaaaattaacattaagaaacagtacaaaatag

SEQ ID NO: 13 - protein, 331 aa, Streptococcus thermophilus
  source 1..331: note = Streptococcus thermophilus clone 11
MATLADIAKLAGVSISTVSRVLNKDETLSVTEDTRHRILTIADEIGYTKYKTINNSKKEKYQVAIIQWVSEEHELDDIYYYNIRLGIEKRAYELDYEMLHFFNDIPSSLGEEVVGVLCIGKFSREQIAKLERLKKTLVFVDSDTLNQGHPCVTTDFENSVQSALCYLKEQGCNNIGLLIGQEKTTDATEIISDPRLRSYRNYCMEKGIYDPLFILTGDFTVQSGYELLDSKIKSGATLPDAYFAASDSLAIGALRALQENGIKVPDDIQIISFNDTTLAKQVYPPLSSVTVYTEEMGRTAMDILNKQLLAPRKIPTLTKLGTKLTLRNSTK

SEQ ID NO: 14 - protein, 331 aa, Streptococcus thermophilus
  source 1..331: note = Streptococcus thermophilus clone 11R15
MATLADIAKLAGVSISTVSRVLNKDETLSVTEDTRHRILTIADEIGYTKYKTINNSKKEKYQVAIIQWVSEEHELDDIYYYNIRLGIEKRAYEPDYEMLHFFNDIPSSLGEEVVGVLCIGKFSREQIAKLERLKKTLVFVDSDTLNQGHPCVTTDFENSVQSALCYLKEQGCNNIGLLIGQEKTTDATEIISDPRLRSYRNYCMEKGIYDPLFILTGDFTVQSGYELLDSKIKSGATLPDAYFAASDSLAIGALRALQENGIKVPDDIQIISFNDTTLAKQVYPPLSSVTVYTEEMGRTAMDILNKQLLAPRKIPTLTKLGTKLTLRNSTK

SEQ ID NO: 15 - DNA, 14 bp, Streptococcus thermophilus
  source 1..14: mol_type = other DNA
tgaaaggggttacg